(19) United States
(12) Patent Application Publication
(10) Pub. No.: US 2013/0011870 A1
(43) Pub. Date: Jan. 10, 2013

(54) Method For Assaying Diseases Characterized By Dyslipidemia

(76) Inventor: Thorsten Hornemann, Zurich (CH)

(21) Appl. No.: 13/553,211

(22) Filed: Jul. 19, 2012

Related U.S. Application Data
(63) Continuation of application No. PCT/EP2011/050660, filed on Jan. 19, 2011.

(30) Foreign Application Priority Data
Jan. 20, 2010 (EP) 10151197.0

(51) Int. Cl. C12Q 1/48

(52) U.S. Cl. 435/15

(57) ABSTRACT

A method for diagnosing a disease or for evaluating the risk to develop a disease which is characterized by dyslipidemia in humans, in particular for diagnosing atherosclerosis, coronary heart disease, peripheral vascular disease, stroke, metabolic syndrome, diabetes (type I and II) and diabetes related sequale (diabetic polyneuropathy, diabetic retinopathie or diabetic nephropathie) as well as lipid associated neuropathies (like Charcot-Marie-Tooth neuropathies such as hereditary sensory and autonomous neuropathy type 1 (HSAN1)) by measurement of atypical products of serine palmitoyltransferase.

### CROSS-REFERENCE TO RELATED APPLICATIONS

The present invention is a continuation of pending International Patent Application PCT/EP2011/050660 filed on Jan. 19, 2011 which designates the United States and claims priority from European Patent Application 10151197.0 filed on Jan. 20, 2010, the content of which is incorporated herein by reference.

### FIELD OF THE INVENTION

The present invention relates to a method for diagnosing a disease or for evaluating the risk to develop a disease which is characterized by dyslipidemia in humans, in particular for diagnosing atherosclerosis, coronary heart disease (CHD), peripheral vascular disease (PVD), stroke, the metabolic syndrome, diabetes (type I and II) and diabetes related sequale (like diabetic polyneuropathy, diabetic retinopathie or diabetic nephropathie) as well as lipid associated neuropathies (like Charcot-Marie-Tooth neuropathies such as hereditary sensory and autonomous neuropathy type 1 (HSAN1)). In this context, the term “dyslipidemia” according to the present invention is not restricted to pathological alterations of cholesterol or triglyceride levels only, but includes the general pathological alterations of one or more type of plasma lipid levels including sterols, triglycerides, phospholipids, sphingolipids and other lipid like structures.

The analysis according to the present invention is based on measuring the levels of minor or atypical products of serine palmitoyltransferase (SPT) such as C14-sphinganine (d14:0), C14-sphingosine (d14:1), C16-sphinganine (d16:0), C16-sphingosine (d16:1), sphinga-diene (d18:2), 1-deoxymethyl-sphinganine (m17:0), 1-deoxymethyl-sphingosine (m17:1), 1-deoxy-sphinganine (m18:0) and 1-deoxy-sphingosine (m18:1) or variants thereof, in body fluids or biopsies. Variants of the invention can be derivates of d14:0, d14:1, d16:0, d16:1, m17:0, m17:1, m18:0, d18:2 and m18:1 especially N-acetylated derivates or derivatives with modifications of other functional and non functional side groups.

The mentioned markers can also be used for monitoring and verifying the efficacy of therapeutic interventions in a disease, e.g. diabetes or atherosclerosis and for the differential diagnosis to exclude the presence of a disease, e.g. the metabolic syndrome or atherosclerosis.

### BACKGROUND OF THE INVENTION

Diseases characterized by dyslipidemia include but are not limited to the metabolic syndrome and diabetes, lipid associated neuropathies (like diabetic sensory polyneuropathy DSN, HS(A)N1, Charcot Marie Tooth) and atherosclerosis (leading to coronary heart disease, peripheral vascular disease and stroke).

Metabolic Syndrome and Diabetes mellitus are characterized by a disorder in carbohydrate and lipid metabolism associated with inappropriately high blood sugar levels (hyperglycemia) resulting from either low levels of the hormone insulin or from abnormal resistance to insulin effects coupled with inadequate levels of insulin secretion. No specific blood marker is currently available for the diagnosis of the metabolic syndrome.

Lipid associated sensory neuropathies are a heterogeneous group of neuropathies which are associated with an impaired carbohydrate or lipid metabolism as it is seen in Diabetes mellitus (type I and II), Hereditary-Sensory-Neuropathy type 1 (HSAN1), Tangier disease, Fabri disease and others.

Atherosclerosis is defined by the accumulation of cholesterol enriched lipid plaques in the in the intima of large blood vessels (e.g. in coronary heart vessels leading to CHD). The disruption of these plaques leads to the formation of thrombi and secondly to the occlusion of vessels resulting in heart infarct, thrombosis or stroke. Today the risk evaluation for the development of atherosclerosis is based on the personal anamnesis of the patient (Gender, Smoking and Diabetes etc.), blood pressure, the levels of cholesterol (total, HDL and LDL) and triglycerides in the plasma.

In general, there is an urgent clinical demand for a prognostic and specific marker for diseases characterized by dyslipidemia which allows early diagnosis and more reliable risk assessment in metabolic disorders like metabolic syndrome, diabetes and atherosclerosis or to assess the risk for developing intricacies like a sensory polyneuropathy or a retinopathy in diabetes.

Products of serine palmitoyltransferase: sphingolipids comprise a family of membrane lipids, such as sphingomyelin and glycosphingolipids and bioactive lipids, such as ceramides, sphingosines, dihydro-sphingosines (sphinganines) or sphingosine-1-phosphate. The first step of the cellular sphingolipid biosynthesis is the condensation of serine and palmitoyl-CoA to form 3-ketosphinganine, a reaction catalyzed by serine-palmitoyltransferase (SPT).

Although the condensation of palmitoyl-CoA with serine is commonly the first step in the de novo synthesis of sphingolipids SPT is not strictly dependent on serine and palmitoyl-CoA as substrates. The enzyme is able to use other amino acids like alanine, glycine and to a certain extent also threonine as alternative substrates. Also the specificity for the activated fatty acid can vary and ranges from acyl-CoA esters with a carbon chain length from 12, 13 or 14 to 18. The conjugation of the various acyl-CoAs with serine forms sphingoid bases with even and odd chain lengths whereas the conjugation of palmitoyl-CoA with alanine and glycine forms the atypical sphingolipids 1-deoxy-sphinganine (doxSA, m18:0) and 1-deoxymethyl-sphinganine (doxmethSA, m17:0), respectively. Sphingoid bases are subsequently N-acetylated by ceramide synthase (CerS) and at least partly metabolized by the ceramide desaturase (DES). The metabolites are therefore found in both, the saturated (e.g. d16:0, m17:0 and m18:0) and unsaturated form (e.g. d16:1, m17:1, d18:2 and m18:1) although for m18:1 the position of the double bound can be different from C⁴⁻⁵ (see FIG. 1).

The use of sphingosine with a C18 carbon chain for the detection of heart disease has been described in U.S. Pat. No. 6,210,976, however not the use of deoxysphingosins, or sphingosins with other carbon chain lengths.

The technical problem underlying the present invention is therefore the provision of a novel means for diagnosing a disease characterized by dyslipidemia.

The solution to the above technical problem is provided by the embodiments of the present invention characterized in the claims.

### SUMMARY OF THE INVENTION

In particular, the present invention relates to the in-vitro use of an atypical product of serine-palmitoyltransferase (SPT) of formula (1a) or (1b)

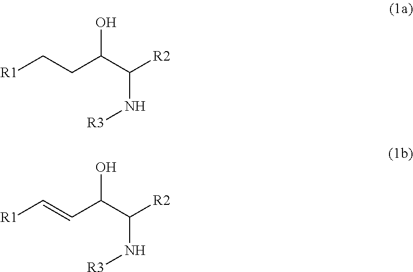

wherein
R₁ represents a group of the formula (—CH₂—)ₙ—CH₃ with n being an integer of 6 to 16, which group may contain one or more C-C double bonds such as, for example in sphingadiene (d18:2);
R₂ is independently selected from the group consisting of hydrogen, methyl and —CH₂—R₄ with R₄ being a hydroxyl, phosphate, phosphocholine or carbohydrate group;
R₃ represents hydrogen or a group of the formula —CO—R₅ wherein R₅ represents a group of the formula (—CH₂—)ₘ—CH₃ with m being an integer of 5 to 25, which latter group may contain one or more C-C double bonds;
with the proviso that said product of serine palmitoyltransferase is not C18-sphinganine (d18:0), C18-dihydroceramide, C18-ceramide, C18-sphingosine (d18:1) or C18-sphingosine-1-phosphate;
for diagnosing a disease characterised by dyslipidemia in a mammal or for evaluating the risk to develop said disease or for monitoring the efficacy of a treatment of said disease, in particular diseases as outlined in the above section “Background of the Invention”.

More specifically, the present invention provides an in vitro method for diagnosing a disease characterised by dyslipidemia in a mammal comprising the steps of:
(a) measuring the level(s) of one or more atypical product(s) of serine-palmitoyltransferase in a sample of said mammal, which product(s) has/have a structure according to formula (1a) or (1b) as defined above;
(b) comparing the level(s) measured in step (a) with the level range(s) of said one or more product(s) in samples of healthy mammals;
wherein a level of said product(s) measured in step (a) being outside of the level range(s) in samples of healthy mammals is indicative of said disease or of having a risk to develop said disease.

The present invention relates to a method for the diagnosis and the monitoring of a disease characterized by dyslipidemia, in particular for diagnosing metabolic syndrome, diabetes, atherosclerosis and lipid related neurological diseases wherein the level of atypical products of serine-palmitoyltransferase as defined above, preferably levels of C14-sphinganine (d14:0), C14-sphingosine (d14:1), C16-sphinganine (d16:0), C16-sphingosine (d16:1), 1-deoxymethyl-sphinganine (m17:0), 1-deoxymethyl-sphingosine (m17:1), 1-deoxy-sphinganine (m18:0) and 1-deoxy-sphingosine (m18:1), sphinga-diene (d18:2) or variants thereof are measured in body fluid samples, biopsies or other human tissues. An altered level is either indicating the presence of the disease, the risk of developing the disease or is representing the stage and progression of the disease. The method is also suitable to monitor the efficacy of a treatment in a disease characterized by dyslipidemia. In this context, the method as described above is preferably repeated during the time of treatment, e.g. monthly (one time, two times or more per month), weekly (one time, two time or more per week) or also daily measurements, especially in severe cases, are envisaged.

In addition, the present invention relates to a method of measuring of d16:0 d16:1, m17:0, m17:1, m18:0 and m18:1 concentration and a kit of parts for such a method.

### BRIEF DESCRIPTION OF THE DRAWINGS

The figures show:

FIG. 1 Chemical structures of some atypical sphingoid bases; A-1-deoxy sphinganine (m18:0); B-1-deoxy sphingosine (m18:1); C-1-deoxymethyl sphinganine (m17:0); D-1-deoxymethyl sphingosine (m17:1); E-C16-sphinganine (d16:0); F-C16-sphingosine (d16:1). An abbreviated nomenclature for sphingoid bases is used in the context of the present invention. The number of hydroxyls are designated by “m” (mono-) and “d” (di-) followed by the number of carbons. The second number indicates the double bonds. For example: d18:0 stands for sphinganine and d18:1 for sphingosine. All shown metabolites are also present in the diacyl form with N-linked fatty acids.

FIG. 2 is a schematic representation of the de-novo synthesis of atypical sphingolipids by serine-palmitoyltransferase (SPT). SPT catalyses the initial conjugation of palmitoyl-CoA with L-serine to form sphinganine. The use of myristoyl-CoA (a) instead of palmitoyl-CoA results in C16-Sphinganine (d16:0). The use of alanine (b) instead of serine, forms 1-deoxy sphinganine (m18:0). These metabolites are acetylated by ceramide synthase (CerS) and desaturated by ceramide desaturase (DES) to form C16-Ceramide or N-acyl-deoxy-sphingosine, respectively. After deacetylation C16-sphingosine and 1-deoxy-sphingosine (m18:1) are formed.

FIG. 3 In vitro SPT activity with various acyl-CoA substrates (C12-CoA, C14-CoA, C16-CoA and C18:1-CoA) in control, SPTLC1, SPTLC2 and SPTLC3 overexpressing HEK293 cells. SPTLC3 overexpressing cells showed a significantly higher activity with lauroyl (C12)- and myristoyl (C14)-CoA forming d14:0 and d16:0-sphingoid bases. The activity with stearoyl and oleoyl-CoA was comparable for all cell lines. (For comparison the activity with palmitoyl-CoA is defined as 100%.)

FIG. 4 Levels of C16-sphinganine (d16:0), C16-sphingosine (d16:1), 1-deoxy sphinganine (m18:0) and 1-deoxy sphingosine (m18:1) in healthy controls (A, n=98) and patients with a coronary heart disease (B, n=89). Concentrations are given in pmol per 100 ul plasma (C). The d16:1 levels were significantly lower in CHD/atherosclerosis patients. Also d16:0 levels were, by trend, lower in the CHD patients whereas m18:1, m18:0 were not significantly different between the two groups.

FIG. 5 Levels of C16-sphinganine (d16:0), C16-sphingosine (d16:1), 1-deoxy sphinganine (m18:0) and 1-deoxy sphingosine (m18:1) in healthy controls (A, n=38) and patients with a clinically diagnosed diabetes (B, n=40). Concentrations are given in pmol/100 ul plasma (C). Deoxy-sphingosine (m18:1) and deoxy-sphinganine (m18:0) levels were significantly elevated in diabetic patients compared to controls.

FIG. 6 shows graphical representations of m18:0 and m18:1 levels in healthy individuals (A), individuals with a stenosis (B) (vessel occlusion >50%), with an metabolic syndrome (C) and with a diagnosed diabetes mellitus (D) (25 individuals each). The m18:0 and m18:1 levels were significantly higher in patients with the metabolic syndrome and diabetes compared to healthy individuals. Also non diabetic, atherosclerotic patients showed elevated m18:0 and m18:1 levels in this cohort but to a lower extent, if compared to patients with the metabolic syndrome or diabetes. Levels are given in pmol per 100 ul plasma (E).

FIG. 7 Box plot representations of m18:0, m18:1 and d16:1 levels in the control (A), metabolic syndrome (MetS) (C) and diabetic (D) group (same study as in FIG. 6). DoxSA and doxSO levels are significantly higher in the MetS and diabetic group were C16SO levels lower in the diabetics but not in control and MetS group. (Box represents the upper and lower quartile, whiskers show the 5% and 95% percentile, the horizontal line represents the median, values given in μM (A))

FIG. 8 (I) Score plots of OPLS-DA models Control vs. MetS (left) and MetS vs. T2DM (right), for example 4. Individual observations are shown as black triangles for controls, open triangles for MetS and black rhombus for T2DM. Clustering of different groups is apparent with acceptable separation. (II) Loading column plots of OPLS-DA models Control vs. MetS (left) and MetS vs. T2DM (right). The weights represent the contribution of each variable to the model component scores. Variables with larger weights contribute more to the model. Error bars represent 95% confidence intervals for calculated weights. (III) Variable importance to the projection (VIP) plot for OPLS-DA models Control (left) vs. MetS and MetS vs. T2DM (right). The VIP coefficients plot shows the summation of all the weights for each X variable to predict Y and hence denoting the “importance” of each X variable. For control vs. MetS; triglycerides, HDL, doxSO and DoxSA can be considered important (y>1) while in the MetS vs. T2DM; glycosylated haemoglobin, glucose and C16SO can be considered important. (IV) Receiver operator characteristics curves (ROC). Triglycerides and HDL cholesterol are considered the gold standard for MetS (left) with AUC's of 0.968 (p<0.001) and 0.111 (0.899 reciprocal), respectively. The DSBs show comparable AUCs with 0.875 for doxSA and 0.842 for doxSO. For T2DM (right) HbA1c and glucose are showing an AUC of 0.939 and 0.917 for T2DM whereas C16SO shows an AUC of 0.282 (0.718 reciprocal)

(Legend: A—control, B—Metabolic Syndrome, C—Diabetes type II, 1—triglycerides, 2—HDL (reciprocal), 3—m18:1, 4—m18:0, 5—waist circumference, 6—glucose, 7—HbA1c, 8—d16:1, a—index, b—sensitivity, c—(1—specificity))

FIG. 9 Levels of C16-sphinganine (d16:0), C16-sphingosine (d16:1), 1-deoxy sphinganine (m18:0) and 1-deoxy sphingosine (m18:1), C18-sphinganine (d18:0), C18-sphingosine (d18:1) and SA-diene (d18:2) in a cohort of apparently healthy individuals. (O-HbA1c<5.6% and TG<1.7 mM; 1—HbA1c<5.6% and TG>1.7 mM; 2—HbA1c is between 5.7-6.4% and TG<1.7 mM; 3—HbA1c is between 5.7-6.4% and TG>1.7 mM; 4—HbA1c>6.5% and TG<1.7 mM; 5—HbA1c>6.5% and TG>1.7 mM1)

FIG. 10 shows graphical representations of m18:0 and m18:1 levels in members of families suffering from the inherited sensory neuropathy—HSAN1. Unaffected and affected members of two families were analyzed, which carry either the HSAN1 causing mutation SPTLC1-C133W (Panel I), SPTLC1-C133Y (Panel II) or V144D (Panel III). In case of the C133W mutation significantly higher m18:0 and m18:1 levels (I left) as well as elevated m17:0 and m17:1 levels (I right) were detected in the plasma of the affected patients (P) compared to unaffected controls (C). Also HSAN1 patient with the SPTLC1-C133Y (II) or the SPTLC1-V144D mutation (III) showed elevated m18:0 and m18:1 plasma levels. m17:0 and m17:1 was not detected in C133Y and V144D carrier. Levels are given in pmol per 100 ul plasma (A)

FIG. 11 Absolute (I) and relative (II) distribution of the sphingoid bases m16:1 (1); m17:1 (2); m18:1 (3); m19:1 (4) and m20:1 (5) in human plasma of 25 healthy donors. Concentrations are given in nM (A)

FIG. 12 Single ion chromatograms of all identified atpycial sphingoid base metabolites in a total lipid extract of SPTLC1, SPTLC2 and SPTLC3 expressing Hek293 cells. Values are given in arbitrary units (A) and retention time is shown in minutes (B)

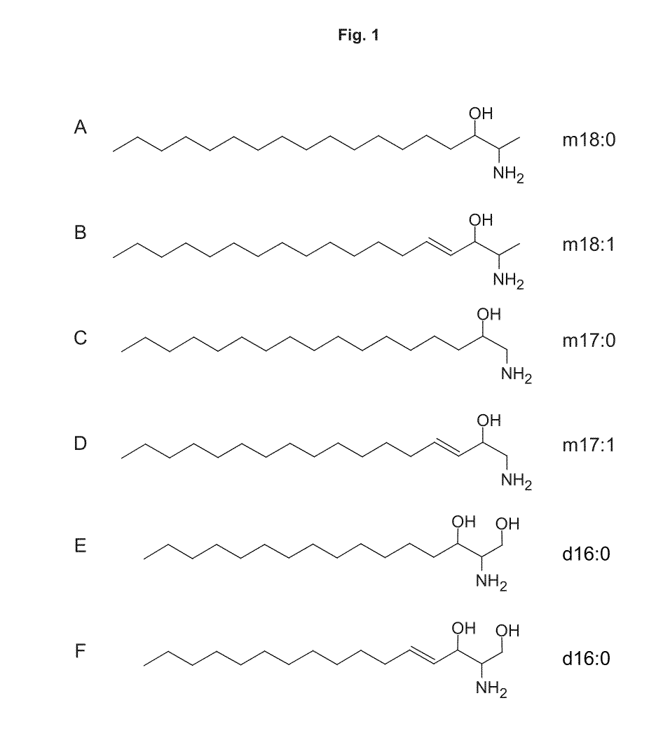

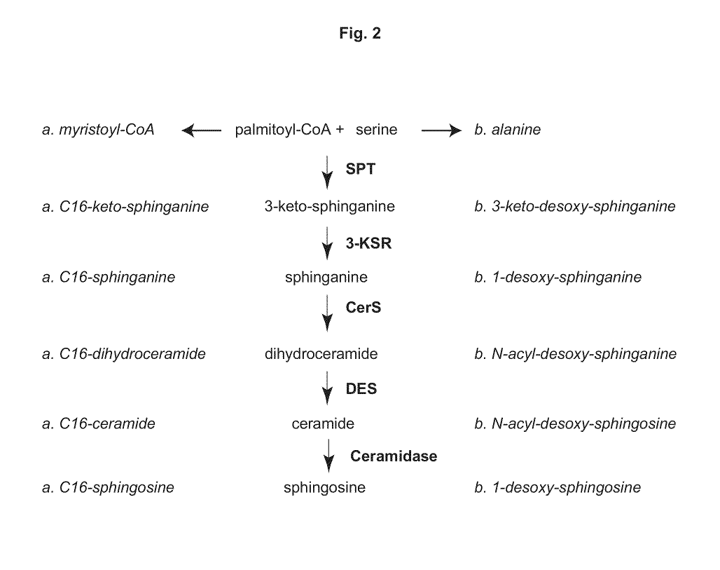

### DETAILED DESCRIPTION OF THE INVENTION

The present invention relates to a method for the diagnosis or risk prediction of a disease characterized by dyslipidemia in mammals, preferably humans, in particular for the diagnosis, monitoring and risk prediction of the metabolic syndrome, diabetes and atherosclerosis, wherein the level of atypical serine palmitoyltransferase products of formula (1a) and (1b) as defined above, in particular of C14-sphinganine (d14:0), C14-sphingosine (d14:1), C16-sphinganine (d16:0), C16-sphingosine (d16:1), 1-deoxymethyl-sphinganine (m17:0), 1-deoxymethyl-sphingosine (m17:1), 1-deoxy-sphinganine (m18:0), sphinga-diene (d18:2) and 1-deoxy-sphingosine (m18:1) or variants thereof is measured in a sample, preferably a body fluid sample, e.g. plasma. The level of one or more atypical SPT products in the sample is compared to the corresponding level(s) in (a) samples of (a) healthy subject(s). An altered level compared to the level in the sample of the healthy subject is indicative of a disease or of the risk to develop the disease.

Also the arithmetic and non-arithmetic combination of atypical products of SPT such as d14:0, d14:1, d16:0, d16:1, m17:0, m17:1, m18:0, d18:2 and m18:1 or variants thereof with each other or with other, either related or non-related risk markers in order to increase the sensitivity, specificity or the predictive value is object of the invention.

Further samples useful for measuring of levels of atypical SPT products as defined herein, in particular d14:0, d14:1, d16:0, d16:1, m17:0, m17:1, m18:0, d18:2 and m18:1 or variants thereof, are e.g. whole blood, serum, urine, sputum, cerebrospinal fluid, tear fluid, sweat, milk, or extracts from solid tissue like biopsies or from fecal matter. The sample can be pre-treated, if desired, and can be prepared in any convenient medium that does not interfere with the measurement. Samples of body fluids can be obtained by any method known in the art.

The level of atypical SPT products such as d16:0, d16:1, m17:0, m17:1, m18:0 and m18:1 or variants thereof, indicative for diseases characterized by dyslipidemia or the risk to develop such disease, is dependent on the type of mammal (e.g. human) and on the sample chosen for the measuring. For plasma (Heparin) the estimated normal levels in humans are:
1-deoxy sphinganine (m18:0): 0.04-0.26 μM*
1-deoxy sphingosine (m18:1): 0.09-0.5 μM*
C16-sphingosine (d16:1) 0.2-0.9 μM*
C16-sphingosine (d16:1) 8-30 μM*
(This range represents the 2.5%-97.5% percentile of a cohort of 100 healthy people; d20:0 is used as internal calibration. Lipids may be analysed according to the method described in Example 1.)

Levels which deviate from these concentrations are indicative for the diseases of the present invention or for the risk of developing such a disease, respectively.

Hence, according to a preferred embodiment, the invention relates to a method of diagnosis of diseases characterized by dyslipidemia, wherein the level of d14:0, d14:1, d16:0, d16:1, m17:0, m17:1, m18:0, d18:2 and m18:1 is measured in a body fluid, and an altered level from the concentrations mentioned above, is indicative of the disease characterized by dyslipidemia or the risk to develop such disease.

In particular, levels of m18:0 and m18:1 being above the concentrations mentioned above are indicative of the metabolic syndrome, diabetes or for a sensory neuropathy like the diabetic polyneuropathy or HSAN1 in humans. Levels of d14:0, d14:1, d16:1 and d16:0 which are below the concentrations mentioned above are indicative of atherosclerosis in humans. An arithmetic combination of several or all markers (e.g. d14:0, d14:1, d16:0, d16:1, m18:0 and m18:1 or d16:0, d16:1, m17:0, m17:1, m18:0, d18:2 and m18:1) is even a more significantly indicative for diabetes and/or atherosclerosis.

Any known method may be used for measuring the levels of atypical products of SPT, particularly d14:0, d14:1, d16:0, d16:1, m17:0, m17:1, m18:0, d18:2 and m18:1 or variants thereof in a sample such as body fluids. Methods considered are e.g. chromatography, mass spectrometry (and combinations thereof), enzymatic assays, electrophoresis and antibody based assays (like ELISA, RIA, EIA, CEDIA, microarray analysis, fully-automated or robotic immunoassays and latex agglutination assays). Such methods, when used for the diagnosis of diseases characterized by dyslipidemia, e.g. metabolic syndrome, diabetes or atherosclerosis, are a further object of the invention.

Furthermore, the person skilled in the art is familiar with the method to measure the level of atypical products of SPT, particularly d14:0, d14:1, d16:0, d16:1, m17:0, m17:1, m18:0, d18:2 and m18:1. The term “level” relates to an amount or concentration of a lipid in an individual or a sample taken from an individual. The term “measuring” and “measured” according to the present invention relates to determining the amount or concentration, preferably semi-quantitatively or quantitatively, of these metabolites, or other substances of interest. Atypical SPT products of particular interest in the context of the present invention are d14:0, d14:1, d16:0, d16:1, m17:0, m17:1, m18:0, d18:2 and m18:1. The levels of d14:0, d14:1, d16:0, d16:1, m17:0, m17:1, m18:0, d18:2 and m18:1 can be measured by quantitative, semi-quantitative and qualitative methods, as well as by all other methods for measuring d14:0, d14:1, d16:0, d16:1, m17:0, m17:1, m18:0, d18:2 and m18:1. For example, a method that merely detects the presence or absence of d16:0, d16:1, m17:0, m17:1, m18:0, d18:2 or m18:1 in a sample suspected of containing these metabolites, is considered to be included within the scope of the present invention. The terms “detecting”, “monitoring” and “determining”, as well as other common synonyms for measuring, are contemplated within the scope of the present invention.

Measuring can be done directly or indirectly. Indirect measuring includes measuring cellular responses, bound ligands, labels, or enzymatic reaction products. In the context of the present invention, amount also relates to concentration. It is evident, that from the total amount of a substance of interest in a sample of known size, the concentration of the substance can be calculated, and vice versa.

Measuring can be done according to any method known in the art. Preferred methods are described in the following.

A preferred method for the measuring of d14:0, d14:1, d16:0, d16:1, m17:0, m17:1, m18:0, d18:2 or m18:1 in human body fluids, e.g. plasma is an LC/MS or LC/MSMS based method (for a preferred protocol see examples 1 to 6).

Another preferred method for measuring d14:0, d14:1, d16:0, d16:1, m17:0, m17:1, m18:0 and m18:1 is the specific chemical modification of d14:0, d14:1, d16:0, d16:1, m17:0, m17:1, m18:0, d18:2 and m18:1 with an enzymatic, colored, fluorescent or otherwise directly (e.g. fluorescent labels or labels presenting a visible color) or indirectly (e.g. developed by a further compound specific for the label) applied compound allowing easy detection and quantification.

Another preferred method is an enzyme-based detection. An enzymatic reaction which specifically metabolizes an atypical product of SPT according to the present invention, preferably d14:0, d14:1, d16:0, d16:1, m17:0, m17:1, m18:0, d18:2 and m18:1, in an untreated or pretreated sample can be coupled directly or indirectly to a chemical, optical, electrochemical or any other suitable detection method (e.g. enzymatic NADP⁺ reduction monitored by fluorescence or by absorption at 340 nm). The measured enzyme activity or changes in the absolute values given by the detection method reflects the concentration of the d14:0, d14:1, d16:0, d16:1, m17:0, m17:1, m18:0, d18:2 and m18:1 metabolite (or derivates thereof) in the sample.

Another preferred method is an ELISA. In one embodiment of the invention, the ELISA for measuring the atypical products of SPT, preferably d16:0, d16:1, m17:0, m17:1, m18:0 and/or m18:1, consists of a sandwich array: Conventional microtiter plates are coated with one type of antibody (“first” antibody”), e.g. a guinea pig polyclonal antibody, directed against the atypical product of SPT such as d14:0, d14:1, d16:0, d16:1, m17:0, m17:1, m18:0, d18:2 or m18:1. The plates are then blocked and the sample or standard is loaded. After the incubation, a different type of antibody (“second” antibody) against the atypical product of SPT, preferably d14:0, d14:1, d16:0, d16:1, m17:0, m17:1, m18:0, d18:2 or m18:1, is applied, e.g. a polyclonal rabbit antibody. A third antibody detecting the particular type of the “second” antibody, e.g. an anti-rabbit antibody, conjugated with a suitable label, e.g. an enzyme for chromogenic detection, is then added. Finally the plate is developed with a substrate for the label in order to detect and quantify the label, being a measure for the presence and amount of the atypical product of SPT such as d14:0, d14:1, d16:0, d16:1, m17:0, m17:1, m18:0, d18:2 or m18:1. If the label is an enzyme for chromogenic detection, the substrate is a color-generating substrate of the conjugated enzyme. The color reaction is then detected in a microplate reader and compared to standards. Suitable pairs of antibodies (“first” and “second” antibody) are any combination of guinea pig, rat, mouse, rabbit, goat, chicken, donkey or horse. This also includes in-vitro generated antibodies or antibody-like molecules obtained by screening methods like phage display or ribosomal display or other proteins with a similar function as antibodies. Preferred are monoclonal antibodies, but it is also possible to use polyclonal antibodies or antibody fragments. Suitable labels are chromogenic labels, i.e. enzymes which can be used to convert a substrate to a detectable colored or fluorescent compound, spectroscopic labels, e.g. fluorescent labels or labels presenting a visible color, affinity label which may be developed by a further compound specific for the label and allowing easy detection and quantification, or any other label used in standard ELISA.

Other preferred methods of measuring are chromatographic methods combined with optical, chemical, electrochemical, potentiometric, nuclear magnetic resonance (NMR) or mass spectrometer based detection, Radioimmunoassay or competitive immunoassay using a single antibody and chemiluminescence detection on automated commercial analytical robots. Microparticle enhanced fluorescence, fluorescence polarized methodologies, or mass spectrometry may also be used directly. Detection devices, e.g. microarrays, are also useful components as readout systems for levels of atypical products of SPT, in particular d14:0, d14:1, d16:0, d16:1, m17:0, m17:1, m18:0, d18:2 or m18:1 levels.

Optionally, the samples, in particular body fluids, can be pretreated. Particularly, the samples can be hydrolysed to release the N-acyl fatty acid and O-linked headgroups for the detection of the free atypical sphingoid bases such as d14:0, d14:1, d16:0, d16:1, m17:0, m17:1, m18:0, d18:2 or m18:1 (for further details see example 1). Other pretreatment options include chemical modifications to improve the detection and/or the sensitivity of the analysis.

The invention further relates to a kit of parts for measuring d14:0, d14:1, d16:0, d16:1, m17:0, m17:1, m18:0, d18:2 or m18:1 for the diagnosis of a disease characterized by dyslipidemia, for example comprising apparatus, reagents and standard solutions of (an) atypical product(s) of SPT, preferably d14:0, d14:1, d16:0, d16:1, m17:0, m17:1, m18:0, d18:2 and/or m18:1. Apparatus considered are e.g. microtiter plates for ELISA (e.g pre-coated ELISA plates and plate covers) or other related devices. Reagents are those reagents particularly developed and designed for measuring atypical products of SPT, preferably d14:0, d14:1, d16:0, d16:1, m17:0, m17:1, m18:0, d18:2 or m18:1. A suitable reagent may be any kind of small molecule or antibody specific for measuring an atypical product of SPT such as d14:0, d14:1, d16:0, d16:1, m17:0, m17:1, m18:0, d18:2 or m18:1. Standards preferably contain d20:0 (C20-sphinganine; e.g. 100 pmol), or d20:1 (C20-sphingosine; e.g. 100 pmol) or isotopic labeled, preferably deuteriated, d18:0 and d18:1. Certain labeled and non-labeled standards can be obtained commercially (e.g. Avanti Polar Lipids Inc, Alabaster, US) whereas some other isotopically labeled standards have to be synthesized according to standard labeling procedures. The kit of parts may contain further hardware, such as columns or pipettes, solutions such as buffers, blocking solutions and other parts like, filters, and colour tables. Optionally, the kit may additionally comprise a user's manual for interpreting the results of any measurement(s) with respect to diagnosing a disease characterized by dyslipidemia, particularly metabolic syndrome, diabetes and atherosclerosis. Such a manual may include information about what measured level corresponds to what kind of disease. In an embodiment of the present invention the kit comprises a user's manual disclosing that, if the level of atypical products of SPT such as d14:0, d14:1, d16:0, d16:1, m17:0, m17:1, m18:0, d18:2 and/or m18:1 are altered from the normal levels (e.g. as mentioned above for human plasma), then the individual is at risk of developing a disease characterized by dyslipidemia and/or disclosing that, if the level of atypical products of SPT such as d16:0, d16:1, m17:0, m17:1, m18:0, d18:2 and/or m18:1 is not altered, then the individual is not at risk of developing a disease characterized by dyslipidemia. Additionally, such user's manual may provide instructions about correctly using the components of the kit for measuring the level(s) of the respective biomarkers.

The present invention also relates to the use of said kit for determining whether an individual is suffering from a disease characterized by dyslipidemia or is at higher risk to develop such a disease, particularly metabolic syndrome, diabetes, atherosclerosis or a lipid related neurological disorder. The present invention further relates to the use of said kits in any of the methods as mentioned above.

The invention also includes the measuring of different markers in combination, simultaneously or non-simultaneously. In particular, the present invention relates to measuring atypical products of SPT, preferably d14:0, d14:1, d16:0, d16:1, m17:0, m17:1, m18:0, m18:1, d18:2 or variants thereof, in combination with each other or with other markers of the metabolic syndrome, diabetes or atherosclerosis. According to the present invention any further markers may be measured in combination with atypical products of SPT such as d14:0, d14:1, d16:0, d16:1, m17:0, m17:1, m18:0, m18:1, d18:2 or variants thereof. Examples for such markers include, but are not limited to, HbA1c, insulin, C-peptide, fasting or spontaneous glucose, fructosamine, cholesterol (free, HDL and LDL), triglycerides, free fatty acids, C-reactive protein (CRP) or any other markers known in the art.

According to the present invention, the measured levels of atypical products of SPT, preferably of d14:0, d14:1, d16:0, d16:1, m17:0, m17:1, m18:0, d18:2 and/or m18:1, indicates whether an individual is suffering from a disease characterized by dyslipidemia or has an increased risk of developing such disease, in particular a metabolic syndrome, diabetes, atherosclerosis or a neuropathy. The terms used in this context, i.e. “non-increased level”, “not elevated level”, “increased levels” and “decreased levels” are known to the person skilled in the art.

The person skilled in the art is able to further determine actual values for the relevant biochemical markers which correspond to these levels. For example, the levels may be assigned according to percentiles of the levels observed in a representative sample of apparently healthy individuals, typically below an age of 50 years (preferably, the sample comprises at least 100, more preferably at least 500, most preferably at least 1000 individuals). E.g., a non-increased level may correspond to the maximum level observed in the 97.5% percentile of healthy individuals. Alternatively, the levels may be determined as “normal ranges” as known in the state of the art.

The levels may also be determined or further refined by studies performed on individuals by comparing levels of atypical products of SPT, e.g. d14:0, d14:1, d16:0, d16:1, m17:0, m17:1, m18:0, d18:2 and m18:1 levels, of apparently healthy individuals with individuals suffering from a diseases characterized by dyslipidemia like metabolic syndrome, diabetes, lipid associated neuropathies or atherosclerosis. Such studies may also allow to tailor the levels according to the type of disease or/and certain patient sub-groups, e.g. elder patients, patients with medication or patients with a certain lifestyle. Guidance on how such studies may be carried out can also be obtained from the examples as disclosed in this specification.

The value of the levels considered as “increased” or “decreased” may also be chosen according to the desired sensitivity or specificity (stringency) of exclusion. The higher the desired sensitivity, the lower is the specificity of exclusion and vice versa. In the above example, the higher the percentile chosen to determine each level, the more stringent is the exclusion criterion, i.e. less individuals would be considered “risk individuals”.

The method according to the present invention also allows the determination of the risk or the likelihood, respectively, of an individual of suffering from a disease characterized by dyslipidemia. According to the present invention, the terms “risk” or “likelihood” relates to the probability of a particular incident to develop a disease characterized by dyslipidemia. The grade of risk can be decreased, non-increased, increased, or highly increased. “Non-increased risk” or “no likelihood” means that there is apparently no risk of suffering from or of developing a disease characterized by dyslipidemia.

The degree of risk is associated with the levels of (an) atypical product(s) SPT, in particular d14:0, d14:1, d16:0, d16:1 m18:0, d18:2 and m18:1 or variants thereof. A non-altered level of (an) atypical product(s) of SPT, preferably d14:0, d14:1, d16:0, d16:1, m17:0, m17:1, m18:0, d18:2 and m18:1 or variants thereof, indicates no increased risk, an altered level of (an) atypical product(s) of SPT, preferably d14:0, d14:1, d16:0, d16:1, m17:0, m17:1, m18:0, d18:2 and m18:1 or variants thereof, indicates an increased risk, and a highly altered level of (an) atypical product(s) of SPT, preferably d14:0, d14:1, d16:0, d16:1, m17:0, m17:1, m18:0, d18:2 and m18:1 or variants thereof, indicates a highly increased risk. In the case of combined measurement of (an) atypical product(s) of SPT, preferably d14:0, d14:1, d16:0, d16:1, m17:0, m17:1, m18:0, m18:1, d18:2 or variants thereof, and other markers of lipid related diseases the risks are calculated analogously.

If the level of (an) atypical product(s) of SPT, preferably d14:0, d14:1, d16:0, d16:1, m17:0, m17:1, m18:0, m18:1, d18:2 or variants thereof, is altered, the patient will undergo a primary treatment or secondary interventions like the initiation of therapeutic life-style changes, drug therapy depending on risk factor constellation (e.g. Statins) and reviews at regular intervals. If the methods according to the present invention indicate an increased or highly increased risk, it will preferably have consequences for the further treatment of the individual.

Variants in the context of the present invention and also embodied in this invention are structural variants of d14:0, d14:1, d16:0, d16:1, m17:0, m17:1, m18:0, d18:2 and m18:1 including derivates with O-linked headgroups or N-linked fatty acids or other linked functional or non functional groups.

The underlying pathomechanism of the observed presence of altered level of atypical products of SPT such as d14:0, d14:1, d16:0, d16:1, m17:0, m17:1, m18:0, m18:1, d18:2 or variants thereof in a disease characterized by dyslipidemia is not entirely clear yet. The metabolites d14:0, d14:1, d16:0, d16:1, m17:0, m17:1, m18:0, d18:2 and m18:1 are atypical products of the enzyme serine palmitoyltransferase (SPT). SPT is a pyridoxal-5-phosphate (PLP) dependent aminotransferase that catalyses the condensation of serine with an activated fatty acid—mostly palmitoyl-CoA. It consists of three subunits (SPTLC1, SPTLC2 and SPTLC3). Both, the SPTLC2 and SPTLC3 subunit bear a PLP binding motive and are involved in catalysis. The SPTLC2 subunit is rather specific for palmitoyl-CoA as a substrate whereas the SPTLC3 subunit shows broader substrate specificity. The SPT reaction is the first and rate limiting step in the de novo synthesis of sphingolipids. Under certain conditions this enzyme can also metabolize other amino acids like L-alanine and glycine forming the products m18:0, m18:1 and m17:0, m17:1 respectively. The metabolites d14:0, d14:1, d16:0 and d16:1 are generated by the conjugation of serine with lauryl (C12) and myristoyl (C14)-acyl-CoA and are lowered in case of a coronary heart disease.

Diabetes is a condition in which the uptake of blood glucose is defective either by an impaired production of the hormone insulin or by a systemic resistance against insulin. The lack of energy forces the cells to use alternative energy sources like from gluconeogenetic amino acids. Therefore, alanine levels are increased under diabetic conditions. Increased alanine blood concentrations due to the glucose-alanin cycle between liver and muscle could increase the generation of the m17:0, m17:1, m18:0 and m18:1 sphingoid bases.

The present invention is further illustrated by the following non-limiting examples.

### EXAMPLES

### Example 1

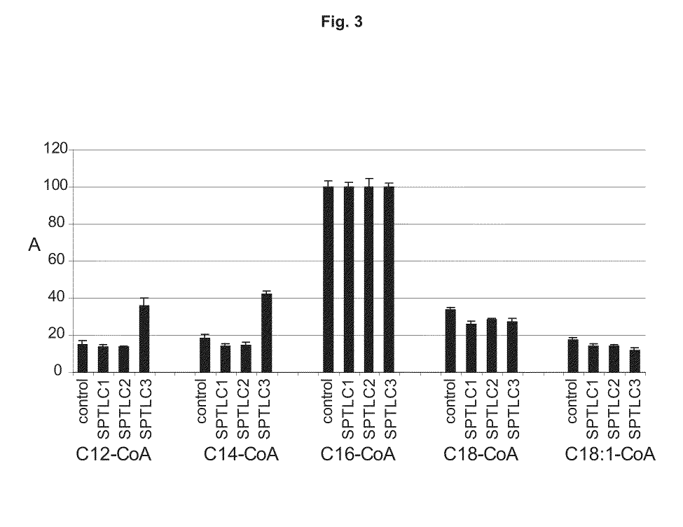

The in-vitro SPT activity was compared for various acyl-CoA substrates in untransfected (control) or SPTLC1-, SPTLC2- and SPTLC3-overexpressing Hek293 cells. All acyl-CoAs were used at the same concentration (50 μM). The SPTLC3 expressing cells showed a significantly higher activity with lauryl-CoA and myristoyl-CoA than compared to the control, i.e. SPTLC1- or SPTLC2-expressing cells (FIG. 3). This suggests that the SPTLC3-mediated SPT activity is primarily responsible for the generation of sphingoid bases with a C14 or C16 backbone whereas the SPTLC2 subunit seems to be more specific for longer acyl-CoAs thereby forming C18 and C20-sphingoid bases.

### Example 2

Plasma was collected from 89 patients which were in hospital for a stent placement and from 98 unaffected persons of the general population.

The samples were centrifuged (3000 g, 15 min) and 100 μl cell free plasma was transferred to a 2 ml polypropylene reaction tube. Lipids were extracted in 1 ml extraction buffer 1 (2 vol. methanol/1 vol. chloroform+0.2 μl/ml C20 SA (1 mM in EtOH). 100 μl of ammonia (2N) was added and the lipids extracted under constant agitation (1 h, 37° C.). Subsequently 0.5 ml chloroform was added and samples were centrifuged (12,000 g, 5 min) to separate the organic from the water phase. The upper (water) phase was removed and the lower phase washed twice with 1 ml of alkaline water (1 ml ammonia (2M) in 100 ml water) and dried under N2.

The dried lipids were resuspended in 200 μl methanolic HCl (1N HCl/10 M water in methanol) and kept at 65° C. for 12-15 hours. The solution was neutralized by the addition of 40 μl KOH (5 M) and subsequently subjected to base hydrolysis. For that 0.5 ml extraction buffer (4 vol. 0.125 M KOH in methanol+1 vol. chloroform) was added and mixed. Subsequently, 0.5 ml chloroform, 0.5 ml alkaline water and 100 μl 2M ammonia was added in this order. Liquid phases were separated by centrifugation (12,000 g, 5 min). The upper phase was aspirated and the lower phase washed twice with alkaline water. Finally, the lipids were dried by evaporation of the chloroform phase under N2 and analysed by LC-MS.

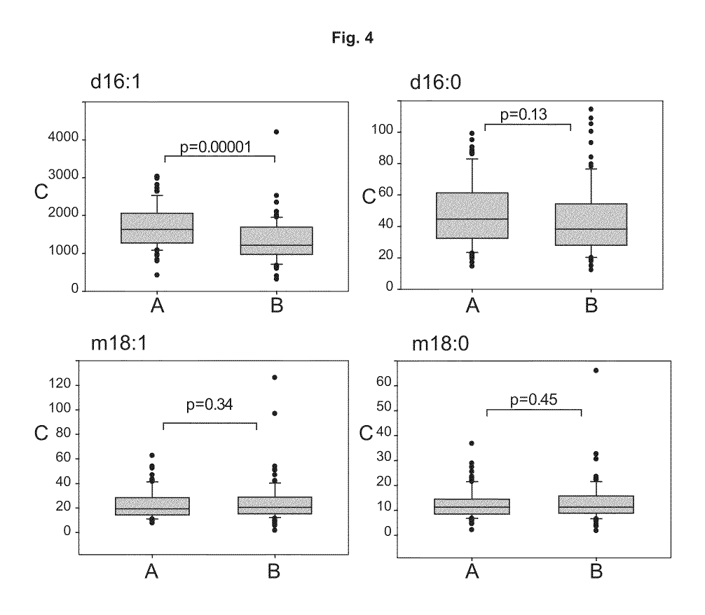

Results of the measurement of d16:1, d16:0, m18:1 and m18:0 are shown in FIG. 4.

### Example 3

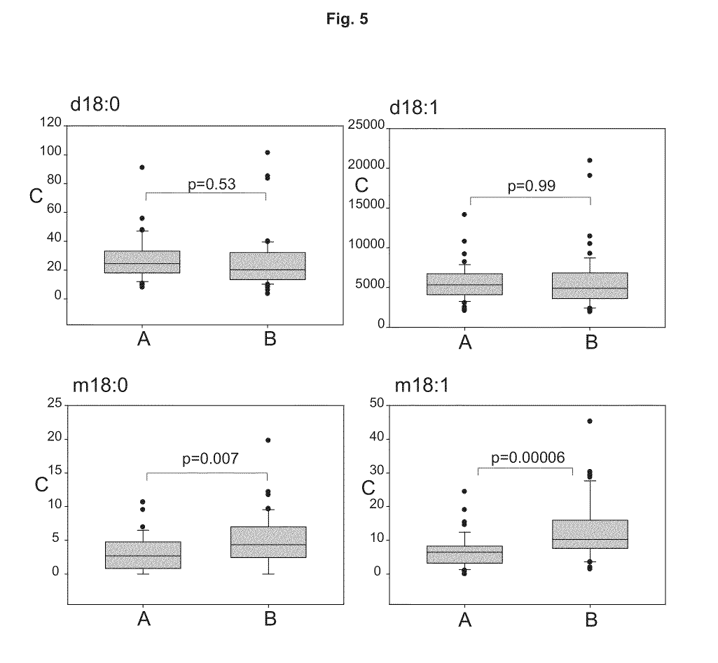

Plasma from 40 persons with diagnosed diabetes was compared with plasma from 38 healthy individuals. Cell free plasma (100 μl) was treated and analyzed as described in Example 2. The m18:0 and m18:1 levels were significantly higher in diabetic patients (B) compared to healthy controls (A) whereas d16:1 and d18:1 levels were not significantly altered between the two groups (cf. FIG. 5).

### Example 4

EDTA Plasma was collected from 25 healthy persons (A), 25 patients with a stenosis (B) (vessel occlusion >50%), 25 persons with an impaired glucose tolerance (metabolic syndrome) (C) and 25 persons with a diagnosed diabetes mellitus (D).

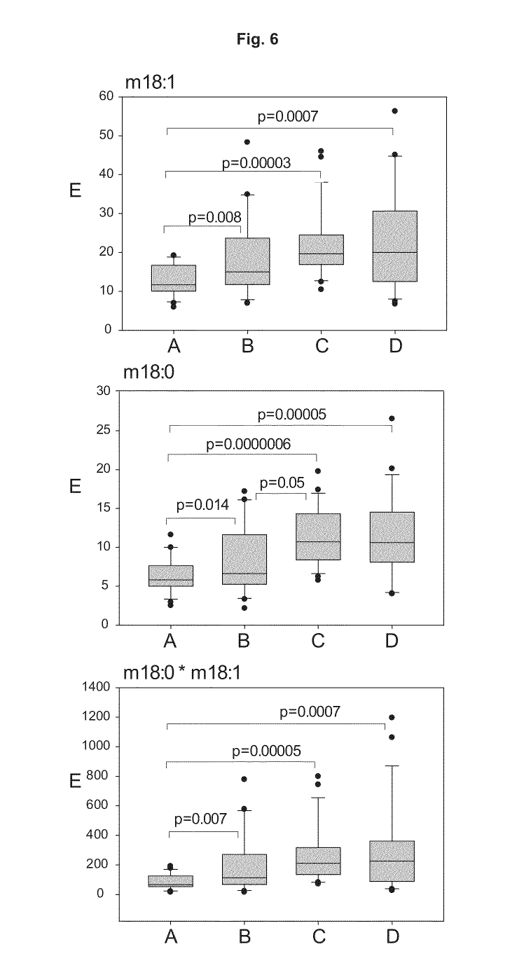

Cell free plasma (100 ul) was treated and analyzed as described in Example 1. The results are shown in FIG. 6. The m18:0 and m18:1 levels were significantly higher in patients with the metabolic syndrome and diabetes. In this cohort also some atherosclerotic patients showed elevated m18:0 and m18:1 levels but less prominent compared to patients with the metabolic syndrome or diabetes.

### Example 5

Plasma samples of patients from three pre-defined subgroups each consisting of 25 sex- and age-matched patients with either manifest diabetes mellitus type 2 (T2DM), metabolic syndrome but no manifest diabetes mellitus (MetS) or healthy controls were analyzed. We examined the plasma concentrations of ten sphingoid base species (C16SO, C16SA, C17SO, C17SA, C18SO, C18SA, C20SO, C18SA-diene, doxSA, and doxSO). C20SA was used as internal standard. Results are summarized in the following Table.

| | | Metabolic | | | |
|---|---|---|---|---|---|
| | | Syndrome | Diabetes | | |
| | Control (n = 25) | (n = 25) | (n = 25) | P-value | P-value |
| | Mean ± SD | Mean ± SD | Mean ± SD | C vs MetS | MetS vs D |
| C16SO (μM) | 8.31 ± 4.49 | 8.38 ± 2.25 | 6.37 ± 2.82 | 0.55 | 0.008 |
| C16SA (μM) | 0.23 ± 0.13 | 0.23 ± 0.10 | 0.26 ± 0.14 | 0.84 | 0.491 |
| C17SO (μM) | 4.66 ± 2.42 | 4.02 ± 1.10 | 3.71 ± 1.48 | 0.64 | 0.29 |
| C17SA (μM) | 0.15 ± 0.07 | 0.14 ± 0.05 | 0.14 ± 0.05 | 0.82 | 0.961 |
| SO (μM) | 88.83 ± 27.23 | 81.66 ± 21.28 | 81.54 ± 23.89 | 0.34 | 0.793 |
| SA (μM) | 2.17 ± 0.9 | 2.20 ± 0.77 | 2.57 ± 1.14 | 0.82 | 0.299 |
| C20SO (μM) | 0.20 ± 0.08 | 0.19 ± 0.06 | 0.19 ± 0.05 | 0.46 | 0.839 |
| doxSO (μM) | 0.15 ± 0.09 | 0.23 ± 0.09 | 0.24 ± 0.13 | 3E−05 | 0.839 |
| doxSA (μM) | 0.06 ± 0.02 | 0.11 ± 0.04 | 0.12 ± 0.05 | 5E−06 | 0.808 |
| C18SA-dienine | 20.79 ± 8.17 | 18.27 ± 5.74 | 16.42 ± 6.80 | 0.36 | 0.273 |
| (μM) | | | | | |
| Female (%) | 40 | 40 | 40 | | |
| AGE | 61.19 ± 4.28 | 62.13 ± 5.02 | 61.56 ± 4.64 | 0.50 | 0.567 |
| Wcf (cm) | 98.24 ± 12.10 | 105.84 ± 8.23 | 110.80 ± 11.97 | 0.004 | 0.051 |
| BMI | 28.19 ± 4.49 | 30.13 ± 3.01 | 32.21 ± 4.83 | 0.05 | 0.09 |
| sysBP (mmHg) | 126.80 ± 12.47 | 133.84 ± 15.23 | 135.52 ± 15.92 | 0.12 | 0.44 |
| diasBP (mmHg) | 78.80 ± 8.50 | 83.92 ± 9.41 | 82.24 ± 8.99 | 0.06 | 0.565 |
| Smoking (%) | 64 | 56 | 60 | | |
| fasting Gluc. (mM) | 5.26 ± 0.56 | 5.64 ± 0.57 | 9.59 ± 4.16 | 0.03 | 4.29E−07 |
| HbA1c (%) | 5.60 ± 0.33 | 5.76 ± 0.34 | 7.79 ± 2.04 | 0.17 | 9.52E−08 |
| Crea | 0.84 ± 0.18 | 0.92 ± 0.21 | 0.81 ± 0.23 | 0.20 | 0.06 |
| AST | 28.56 ± 8.60 | 30.84 ± 24.10 | 25.84 ± 8.43 | 0.38 | 0.662 |
| ALT | 29.92 ± 15.71 | 34.76 ± 24.54 | 31.68 ± 13.25 | 0.35 | 0.907 |
| GFR | 100.65 ± 11.53 | 95.26 ± 15.30 | 100.77 ± 15.68 | 0.22 | 0.184 |
| Chol(mM) | 5.26 ± 1.24 | 4.99 ± 1.30 | 4.98 ± 1.44 | 0.49 | 0.861 |
| LDL(mM) | 3.35 ± 0.96 | 3.45 ± 1.00 | 3.16 ± 1.22 | 0.52 | 0.269 |
| HDL(mM) | 1.80 ± 0.69 | 1.15 ± 0.21 | 1.31 ± 0.32 | 2.37E−06 | 0.132 |
| TG(mM) | 1.10 ± 0.37 | 2.51 ± 0.83 | 2.21 ± 1.47 | 1.38E−08 | 0.067 |

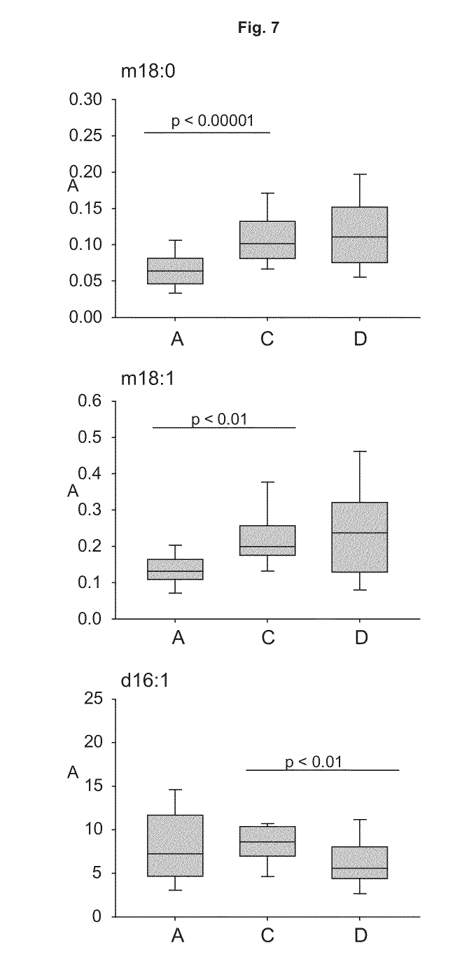

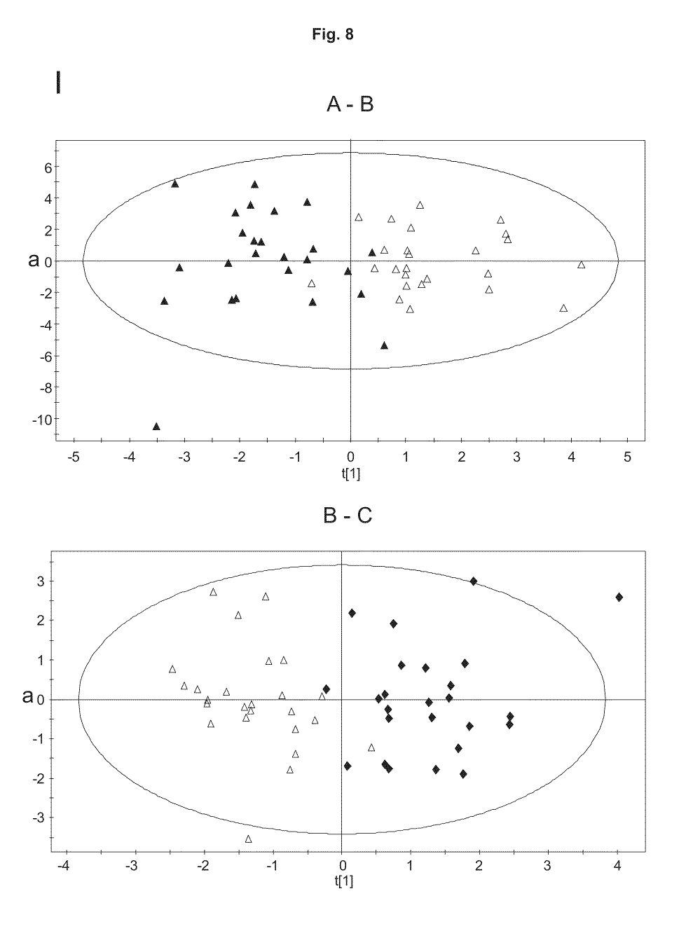

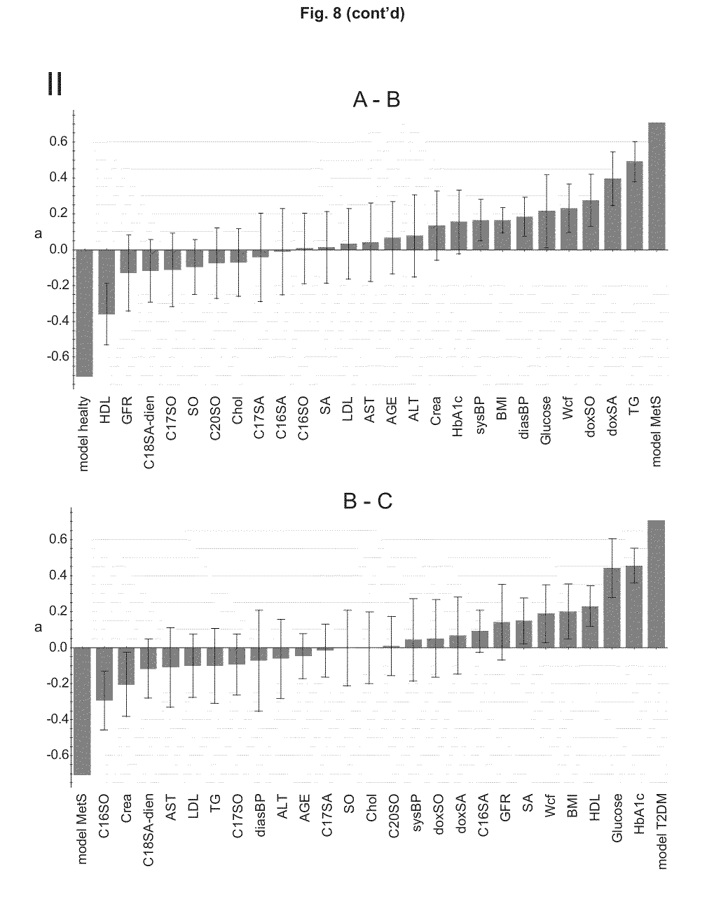

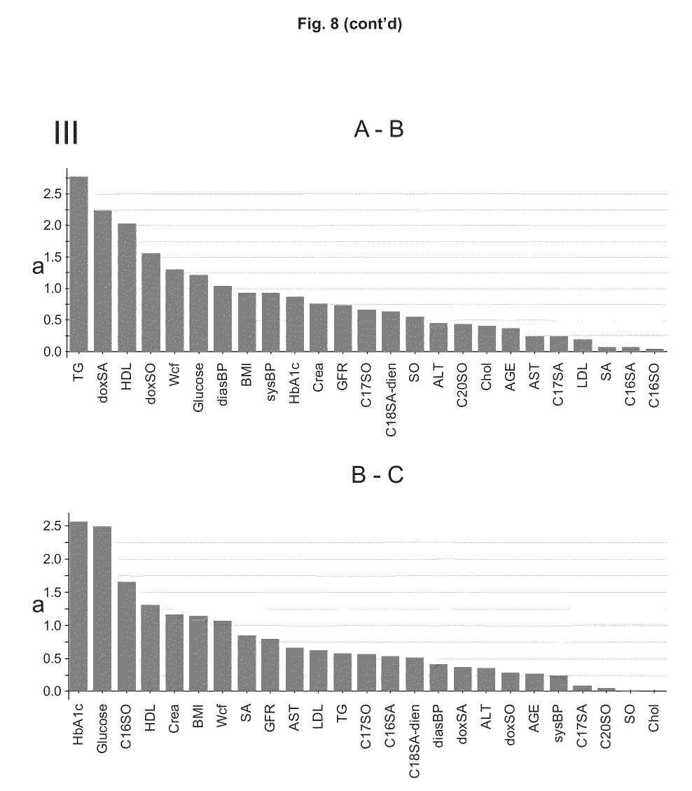

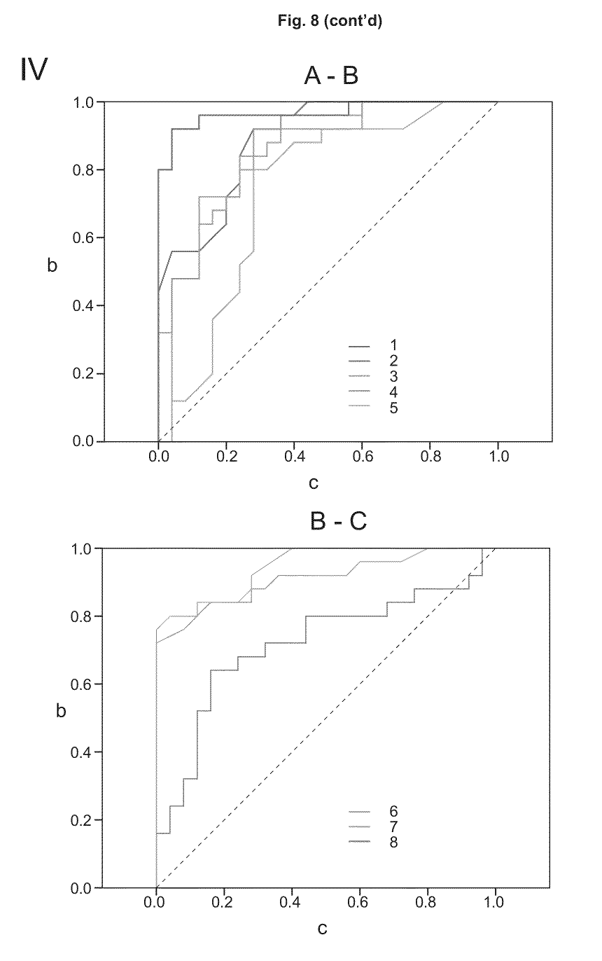

DSB levels are compared to those of patients with a diagnosed stenosis (>50%, FIG. 6). Plasma concentrations of the deoxy-sphingoid bases (DSB) were significantly higher in MetS and T2DM patients compared to controls but did not differ between the MetS and T2DM groups (FIGS. 6 and 7). DSB levels were also significantly elevated in patients with a known stenosis (FIG. 6). In contrast, C16SO levels were found to be significantly lower in T2DM patients in comparison to controls and MetS patients but not different between these two latter groups (FIG. 7). The other sphingoid bases were not significantly different between the three groups. An orthogonal partial least square-discriminant analysis (OPLS-DA) was used to estimate the importance of the individual variables as discriminating biomarkers. Both models (control vs. MetS and MetS vs. T2DM) showed good explanations of the two states and therefore, are valid for discrimination. For both disease states, clustering is still preserved after reducing the dimensions into a single principal component and an orthogonal one (FIG. 8 I). Loading plots (FIG. 8 II) show the weights of each of the variables to the model and hence its contribution to the disease state. In the control vs. MetS loading plot, triglycerides, DoxSA (m18:0) and DoxSO (m18:1), diastolic and systolic blood pressure have the largest contribution to the MetS state model whereas elevated HDL cholesterol showed the largest contribution to the control state model. In the MetS vs T2DM model we found elevated fasting glucose and glycated haemoglobin (HbA1c) concentrations together with low C16SO (d16:1) and creatinine levels to be the largest contributors for the diabetes state model (FIG. 81I). Variable importance for the projection (VIP) plots (FIG. 8 III) shows the contribution of each variable to the variation in both the X space and the Y space. A coefficient value >1 signifies that the variable is “important”. For the control vs. MetS model highest VIP coefficients were seen for triglycerides, DoxSA (m18:0), HDL cholesterol and DoxSO (m18:1). For the MetS vs. T2DM model a significant importance of HbA1c, glucose and C16SO (d16:1) was seen. The diagnostic potential of these markers was analyzed in a receiver operator curve (ROC) analysis (FIG. 8 IV). For the diagnosis of MetS, doxSA and doxSO had an area under the curve (AUC) of 0.875 and 0.842 respectively (p<0.001). C16SO showed an AUC of 0.282 (corresponding to 0.718; p<0.01).

### Example 6

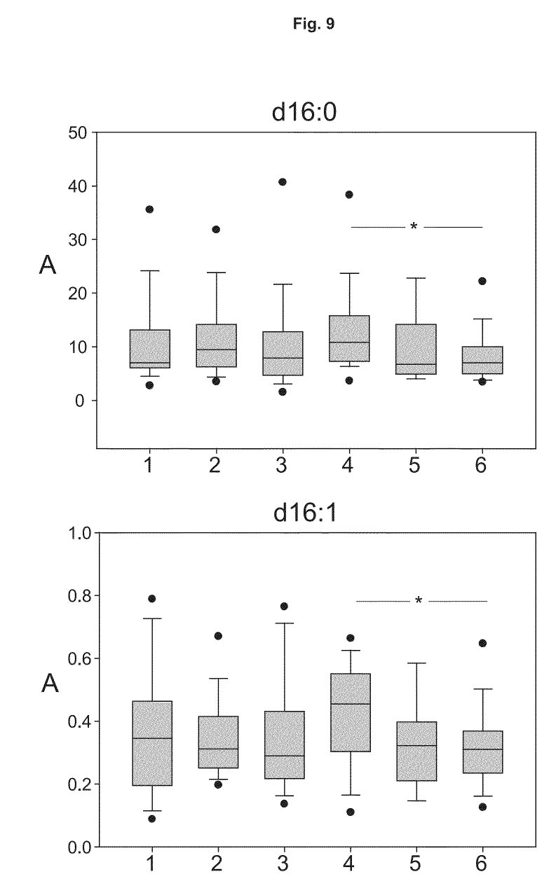

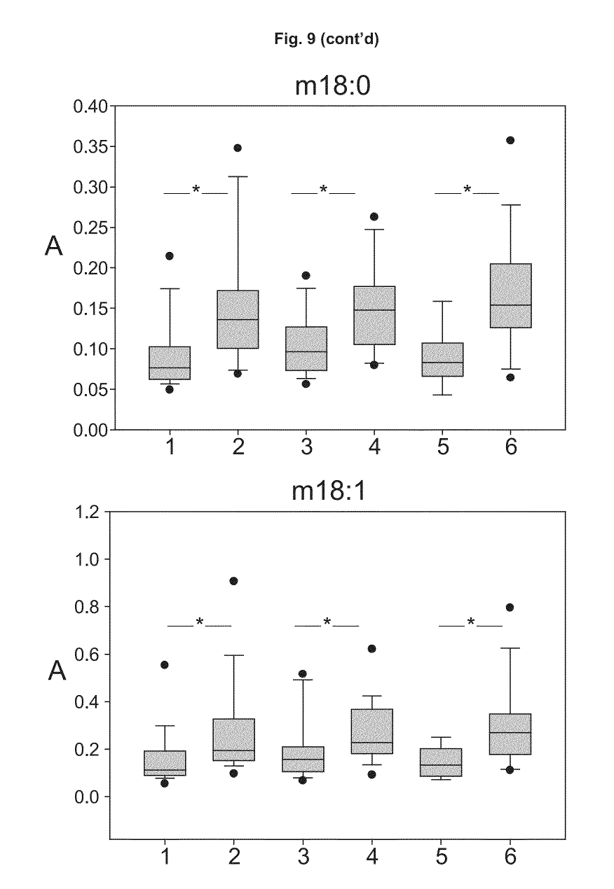

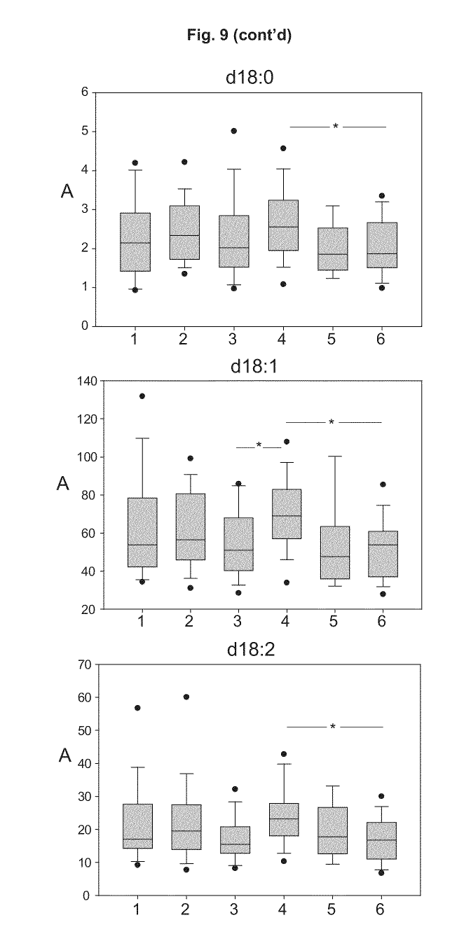

Plasma samples from apparently healthy individuals were subgrouped according to their glacyted hemoglobin (HbA1c) and triglyceride (TG) levels (non fasting) (FIG. 9). We examined the plasma concentrations of C16SO (d16:1), C16SA (d16:0), C18SO (d18:1), C18SA (d18:0), C18SA-diene (d18:2), DoxSA (m18:0), and DoxSO (m18:1). Elevated TG levels are closely associated with increased m18:0 and m18:1 levels.

### Example 7

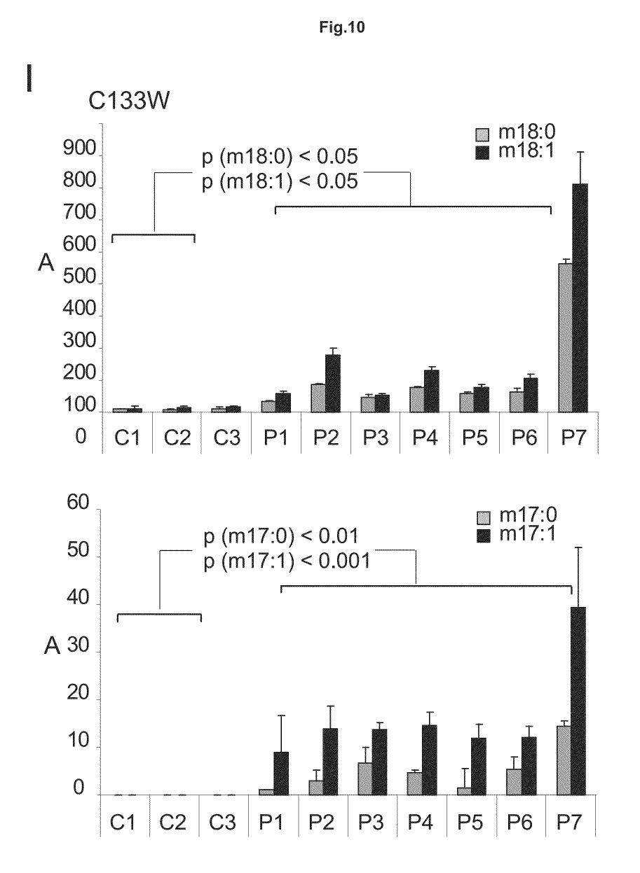

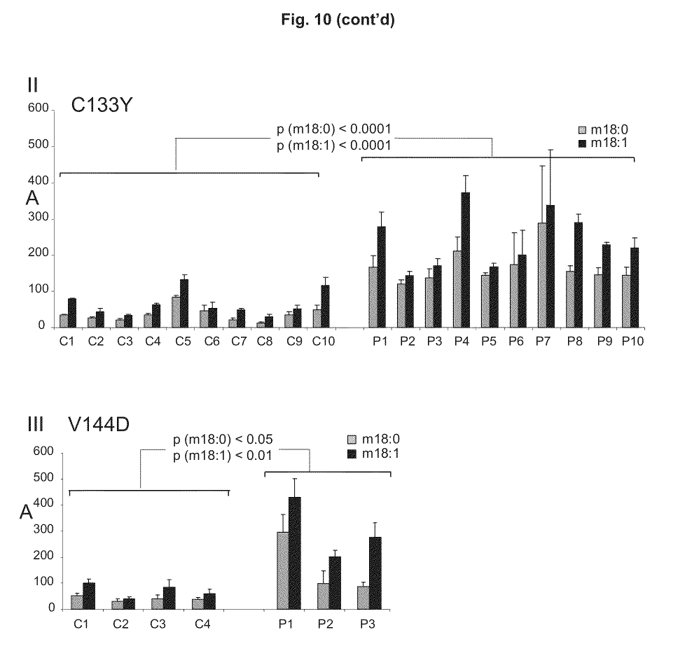

EDTA Plasma was collected from families suffering from mutations in the SPTLC1 gene leading to the hereditary neuropathy HSAN1. The affected members (P) of the first family were carriers of the SPTLC1-C133W (FIG. 10, panel I), the SPTLC1-C133Y (FIG. 10, panel II) or the SPTLC1-V144D mutation (FIG. 10, panel III). All analyses were done from 100 μl cell free plasma. Samples were treated and analyzed as described in Example 1. In all cases the affected patients (P) showed significantly higher m18:0 and m18:1 levels compared to the unaffected controls (C).

### Example 8

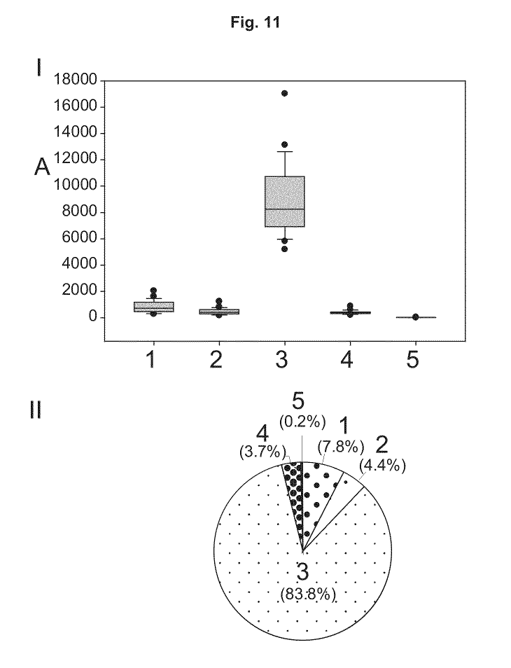

EDTA plasma from 25 healthy donors was extracted according to the method described in Example 1 and plasma levels analysed for (1) d16:1, (2) d17:1, (3) d18:1, (4) d19:1 and (5) d20:1. Values in FIG. 11 are given in absolute concentrations (FIG. 11, panel I: A=pM) and the relative proportion (FIG. 11, panel II).

### Example 9

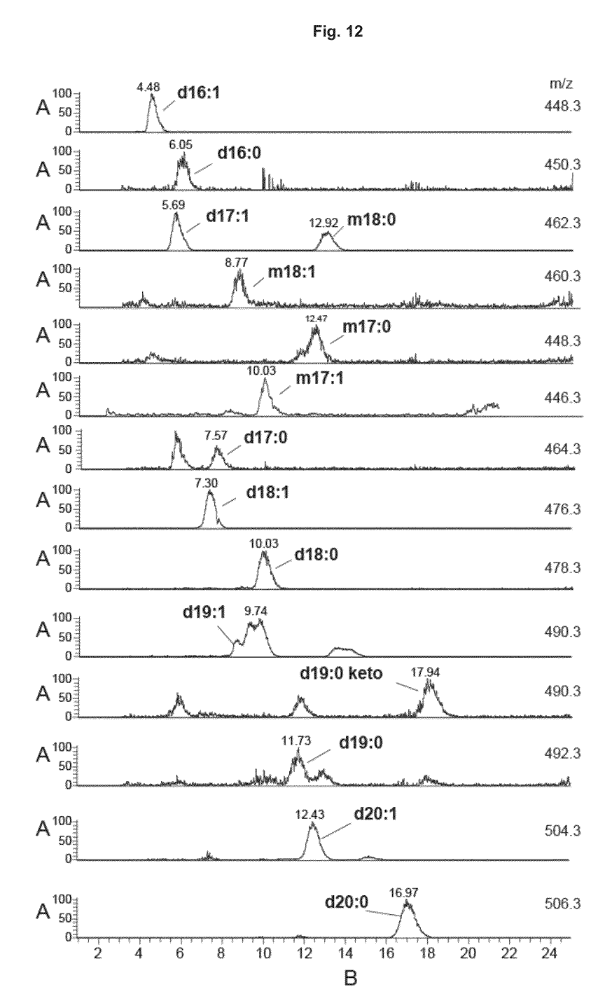

Serine palmitoyltransferase overexpressing HEK 293 cells were analyzed for the presence of atypical sphingoid bases. The single ion chromatograms of all identified aSL metabolites is shown in FIG. 12. The metabolites were derivatized with ortho-phthalaldehyde (OPA), separated on a C18 reverse phase column and analyzed by a tandem arrangement of fluorescence and MS detector. The numbers on the right show the mass to charge ratios (m/z) for the derivatized sphingoid base metabolites (the mass differences between derivatized and underivatized metabolites is 177 Da). Peak intensities are given in arbitrary units (A). The retention time is given in minutes (B).

What is claimed is:

1. In-vitro use of an atypical product of serine palmitoyltransferase of formula (1a) or (1b) 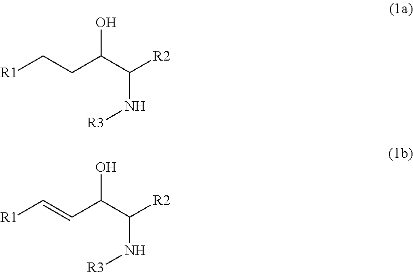
wherein R₁ represents a group of the formula (—CH₂—)ₙ—CH₃ with n being an integer of 6 to 16, which group may contain one or more C—C double bonds;
R₂ is independently selected from the group consisting of hydrogen, methyl and —CH₂—R₄ with R₄ being a hydroxyl, phosphate, phosphocholine or carbohydrate group;
R₃ represents hydrogen or a group of the formula —CO—R₅ wherein R₅ represents a group of the formula (—CH₂—)ₘ—CH₃ with m being an integer of 5 to 25, which latter group may contain one or more C—C double bonds;
wherein said product of serine palmitoyltransferase is not C18-sphinganine, C18-dihydroceramide, C18-ceramide, C18-sphingosine or C18-sphingosine-1-phosphate;
for diagnosing a disease characterised by dyslipidemia in a mammal or for evaluating the risk to develop said disease or for monitoring the efficacy of a treatment of said disease.

2. The use of the atypical product of serine palmitoyltransferase of formula (1a) or (1b) of claim 1 wherein the atypical product of palmitoyltransferase is selected from the group consisting of C14-sphinganine (d14:0), C14-sphingosie (d14:1), C16-sphinganine (d16:0), C16-sphingosine (d16:1), 1-deoxymethyl-sphinganine (m17:0), 1-deoxymethyl-sphingosine (m17:1), 1-deoxy-sphinganine (m18:0), sphinga-diene (d18:2) and 1-deoxysphingosine (m18:1).

3. The use of the atypical product of serine palmitoyltransferase of formula (1a) or (1b) of claim 1 wherein the disease characterised by dyslipidemia is selected from the group consisting of metabolic syndrome, diabetes mellitus, lipid-associated neuropathies and atherosclerosis.

4. The use of the atypical product of serine palmitoyltransferase of formula (1a) or (1b) of claim 3 wherein the diabetes mellitus is of type I or type II.

5. The use of the atypical product of serine palmitoyltransferase of formula (1a) or (1b) of claim 3 wherein the lipid-associated neuropathy is selected from the group consisting of Charcot-Marie-Tooth neuropathies.

6. The use of the atypical product of serine palmitoyltransferase of formula (1a) or (1b) of claim 5 wherein the neuropathy is hereditary sensory and autonomous neuropathy type I (HSAN1).

7. The use of the atypical product of serine palmitoyltransferase of formula (1a) or (1b) of claim 1 wherein the mammal is a human.

8. An in vitro method for diagnosing a disease characterised by dyslipidemia in a mammal comprising the steps of:
a. measuring the level(s) of one or more atypical product(s) of serine palmitoyltransferase of formula (1a) or (1b) in a sample of said mammal: 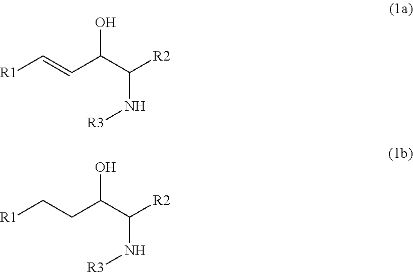
wherein:
R₁ represents a group of the formula (—CH₂—)ₙ-CH₃ with n being an integer of 6 to 16, which group may contain one or more C—C double bonds;
R₂ is independently selected from the group consisting of hydrogen, methyl and —CH₂—R₄ with R₄ being a hydroxyl, phosphate, phosphocholine or carbohydrate group;
R₃ represents hydrogen or a group of the formula —CO—R₅ wherein R₅ represents a group of the formula (—CH₂—)ₘ—CH₃ with m being an integer of 5 to 25, which latter group may contain one or more C—C double bonds;
wherein said product of serine palmitoyltransferase is not C18-sphinganine, C18-dihydroceramide, C18-ceramide, C18-sphingosine or C18-sphingosine-1-phosphate;
b. comparing the level(s) measured in step (a) with the level range(s) of said one or more product(s) in samples of healthy mammals;
c. wherein a level of said product(s) measured in step (a) being outside of the level range(s) in samples of healthy mammals is indicative of said disease or of having a risk to develop said disease.

9. The method of claim 8 wherein the atypical product of palmitoyltransferase is selected from the group consisting of C14-sphinganine (d14:0), C14-sphingosie (d14:1), C16-sphinganine (d16:0), C16-sphingosine (d16:1), 1-deoxymethyl-sphinganine (m17:0), 1-deoxymethyl-sphingosine (m17:1), 1-deoxy-sphinganine (m18:0), sphinga-diene (d18:2) and 1-deoxysphingosine (m18:1).

10. The method of claim 9 wherein the disease characterised by dyslipidemia is selected from the group consisting of metabolic syndrome, diabetes mellitus, lipid-associated sensory neuropathies and atherosclerosis.

11. The method of claim 10 wherein a level of said product(s) above the level range(s) in healthy mammals is indicative of metabolic syndrome, diabetes mellitus or lipid-associated sensory neuropathies of having a risk to develop metabolic syndrome, diabetes mellitus or lipid-associated sensory neuropathies.

12. The method of claim 11 wherein the diabetes mellitus is of type I or type II.

13. The method of claim 11 wherein the lipid-associated neuropathy is selected from the group consisting of Charcot-Marie-Tooth neuropathies.

14. The method of claim 13 wherein the neuropathy is hereditary sensory and autonomous neuropathy type I (HSAN1).

15. The method of claim 10 wherein a level of said product(s) below the level range(s) in healthy mammals is indicative of atherosclerosis or of having a risk to develop atherosclerosis.

16. The method of claim 11 wherein the mammal is a human.

17. The method of claim 8 wherein the disease characterised by dyslipidemia is selected from the group consisting of metabolic syndrome, diabetes mellitus, lipid-associated sensory neuropathies and atherosclerosis.

18. The method of claim 17 wherein a level of said product(s) above the level range(s) in healthy mammals is indicative of metabolic syndrome, diabetes mellitus or lipid-associated sensory neuropathies of having a risk to develop metabolic syndrome, diabetes mellitus or lipid-associated sensory neuropathies.

19. The method of claim 18 wherein the diabetes mellitus is of type I or type II.

20. The method of claim 18 wherein the lipid-associated neuropathy is selected from the group consisting of Charcot-Marie-Tooth neuropathies.

21. The method of claim 20 wherein the neuropathy is hereditary sensory and autonomous neuropathy type I (HSAN1).
